(19) United States
(12) Patent Application Publication
(10) Pub. No.: US 2026/0248554 A1
(43) Pub. Date: Aug. 27, 2026

(54) JAW MEMBER OF INSTRUMENT END EFFECTOR AND RELATED DEVICES, SYSTEMS AND METHODS

(71) Applicant: INTUITIVE SURGICAL OPERATIONS, INC., Sunnyvale, CA (US)

(72) Inventors: Stuart Taylor, San Jose, CA (US); Adrit Lath, Mountain View, CA (US); Scott E. Manzo, Shelton, CT (US)

(73) Assignee: INTUITIVE SURGICAL OPERATIONS, INC., Sunnyvale, CA (US)

(21) Appl. No.: 18/879,438

(22) PCT Filed: Jun. 29, 2023
(86) PCT No.: PCT/US2023/069346

§ 371 Date: Dec. 27, 2024

Related U.S. Application Data
(60) Provisional application No. 63/357,103, filed on Jun. 30, 2022.

(51) Int. Cl. A61B 18/14; A61B 17/00; A61B 17/29; B25J 15/00

(52) U.S. Cl. CPC A61B 18/1447; B25J 15/0028; A61B 2017/00526; A61B 2017/2936; A61B 2017/2947

(57) ABSTRACT

An instrument comprises a shaft and an end effector coupled to the shaft. The end effector comprises a first jaw member and a second jaw member opposing each other, each of the first and second jaw members extending distally relative to the shaft and movable relative to one another between an open configuration and a closed configuration. Each of the first and second jaw members comprises a jaw base and an electrode coupled to the jaw base. A distal portion of the electrode extend distally beyond the jaw base, and each jaw member comprises a support feature configured to support the distal portion of the electrode.

### CROSS-REFERENCE TO RELATED APPLICATION

This application claims priority to U.S. Provisional Application No. 63/357,103 (filed Jun. 30, 2022), titled “JAW MEMBER OF INSTRUMENT END EFFECTOR AND RELATED DEVICES, SYSTEMS AND METHODS” the entire contents of which are incorporated by reference herein.

### FIELD

Aspects of this disclosure relate generally to instrument end effectors and related devices, systems, and methods, for example, for use in computer-assisted teleoperated manipulator systems. More specifically, aspects of the disclosure relate to instrument end effectors having jaw mechanisms, and remotely-controlled instruments including such end effectors.

### INTRODUCTION

Remotely-controlled instruments, which may include non-medical instruments (e.g., industrial instruments) and medical instruments (e.g., surgical instruments, imaging instruments, diagnostic instruments, therapeutic instruments, etc.), generally comprise end effectors, which are often disposed at a distal end portion of the instrument and comprise one or more functional components, such as, for example, a jaw mechanism, a stapler, a knife, a camera, an electrode, a sensor, and various other tools and/or, to perform one or more functions of the instrument, such as, for example, cutting, sealing, grasping, imaging, and various other functions. The functions performed by an end effector may be controlled and driven by mechanical forces and/or other inputs (e.g., electrical energy, illumination, fluid delivery and/or evacuation) received by the instrument via various interfaces generally located at a proximal end portion of the instrument. In some such instruments, actuation elements and/or other functional delivery elements (e.g., fluid or pressure delivery conduits, electrical conduits, data conduits) run from the proximal end portion along an instrument shaft to transmit forces and/or other functionality from a transmission mechanism at the proximal end portion of the instrument to the end effector. Such remotely-controlled instruments can be manually operated, for example, via one or more manually-actuated inputs at a handle or other interface mounted at the proximal end portion. Alternatively, such remotely-controlled instruments may be coupled to or configured to be coupled to computer-assisted manipulator systems, which may be operably coupled to a remotely located console that provides the interface to receive input from a user.

Various types of end effectors, such as forceps, vessel sealers, and staplers, for example, comprise jaw mechanisms. The jaw mechanism comprises a pair of jaw members that are pivotable between open and closed configurations, for example to grasp an object and/or perform other operations on the object. In some end effectors, the jaw mechanism may also include additional functional elements, such as electrodes for electrosurgical functions. Furthermore, some end effectors may include a movable component configured for translational movement relative to the jaw mechanism, such as a cutting component or a staple firing mechanism.

In various applications, the end effector of an instrument is used in workspaces with relatively limited space. For example, in the context of medical instruments, the workspace may comprise a portion of a patient's body and the end effector and shaft may be inserted into the workspace via an incision or natural orifice. Thus, it is generally desirable to provide instruments, and in particular the end effectors thereof, that are relatively small and able to be used and manipulated within the relatively space-constrained regions found in various applications. For example, an end effector with a smaller diameter may allow for less collateral tissue damage to occur as a result of insertion through the opening (e.g., a smaller incision may be made). As another example, if some components of the end effector can be made to take up less space, this may also allow for additional components to be included within the end effector without increasing the overall size thereof, thus expanding the capabilities of the instrument.

As sizes of instruments become smaller, however, challenges arise in their manufacture. Techniques that are relatively more complicated and costly and/or materials that are relatively more costly may be needed in order to produce components that are smaller but still able to effectively perform the functions of the instrument. Moreover, decreasing sizes of end effectors may affect the overall strength, rigidity, and/or stability of certain components thereof, such as of jaw members of a jaw mechanism.

Accordingly, a need exists to provide end effectors with relatively smaller jaw mechanisms which are also relatively simple and cost effective to manufacture, and/or to otherwise improve performance of instrument end effectors.

### SUMMARY

Various embodiments of the present disclosure may solve one or more of the above-mentioned problems and/or may demonstrate one or more of the above-mentioned desirable features. Other features and/or advantages may become apparent from the description that follows.

In accordance with an embodiment, an instrument comprises a shaft and an end effector coupled to the shaft. The end effector comprises a first jaw member and a second jaw member opposing each other, each of the first and second jaw members extending distally relative to the shaft and movable relative to one another between an open configuration and a closed configuration. Each of the first and second jaw members comprises a jaw base and an electrode coupled to the jaw base. A distal portion of the electrode extend distally beyond the jaw base, and each jaw member comprises a support feature configured to support the distal portion of the electrode.

In accordance with another embodiment, an instrument comprises a shaft comprising a proximal end portion and a distal end portion and an end effector coupled to the distal end portion of the shaft. The end effector comprises a first jaw member and a second jaw member opposing and pivotably coupled to each other by pivot pin members and an actuation link engaged with the first and second jaw members to drive the first and second jaw members to pivotably move between open and closed states. Each of the first and second jaw members comprises a long tang and a short tang, the long tang being longer than the short tang. Each of the long and short tangs comprises a pivot aperture engaged with one of the pivot pin members. The long tang comprising a ramped slot engaged with the actuation link.

In accordance with yet another embodiment, an instrument end effector comprises a first jaw member, a second jaw member, and an actuation link. The actuation link comprises a body and pin members extending laterally in opposite directions from the body. Each of the first and second jaw members comprises two pivot apertures configured to receive a pivot pin member, and a ramped slot configured to receive one of the pin members of the actuation link. The first and second jaw members are configured such that the pin members of the actuation link, while coupled to the body, are insertable into respective ramped slots of the first and second jaw members simultaneously during assembly of the first and second jaw members with the actuation link.

In accordance with still another embodiment, an instrument comprises a shaft comprising a proximal end portion and a distal end portion; and an end effector coupled to the distal end portion of the shaft. The end effector comprises a first jaw member and a second jaw member opposing and pivotably coupled to each other by pivot pin members, and an actuation link engaged with the first and second jaw members to drive the first and second jaw members to pivotably move between open and closed states.

Each of the first and second jaw members comprises a jaw base, a long tang and a short tang coupled and extending proximally from the jaw base, long tang being longer than the short tang, an electrode coupled to the jaw base and having a distal portion extending distally be-yond the jaw base, and a support feature configured to support the distal portion of the electrode. Each of the long and short tangs comprises a pivot aperture engaged with one of the pivot pin members. The long tang comprising a ramped slot engaged with the actuation link.

In accordance with another embodiment, a method of assembling a jaw mechanism of an instrument end effector comprises providing a first jaw member comprising two first pivot apertures and a first ramped slot and providing a second jaw member comprising two second pivot apertures and a second ramped slot. The method further comprises coupling an actuation link to an actuation element, the actuation link comprising two pin members extending laterally in opposite directions, and engaging the pin members of the actuation links with the first and second ramped slots by moving the first and second jaw members laterally towards one another. The method further comprises pivoting the first and second jaw members relative to one another while the pin members of the actuation link are engaged with the first and second ramped slots so as to bring the respective first and second pivot apertures of the first and second jaw members into alignment with one another. The method further comprises inserting pivot pin members through the aligned first and second pivot apertures of the first and second jaw members.

### BRIEF DESCRIPTION OF THE DRAWINGS

The present disclosure can be understood from the following detailed description, either alone or together with the accompanying drawings. The drawings are included to provide a further understanding of the present disclosure and are incorporated in and constitute a part of this specification. The drawings illustrate one or more embodiments of the present teachings and together with the description explain certain principles and operation. In the drawings:

FIG. 1 is a schematic view of an embodiment of an instrument.

FIG. 2 is a schematic side view of an embodiment of a jaw mechanism that may be used in the instrument of FIG. 1.

FIG. 3A is a side view of jaw members of another embodiment of a jaw mechanism that may be used in the instrument of FIG. 1.

FIG. 3B is a cross-section of the jaw members of FIG. 3A, with the section taken along 3B-3B in FIG. 3A.

FIG. 4 a front, side perspective view of an embodiment of an end effector comprising a jaw mechanism.

FIG. 5 is an exploded view of a jaw member of the end effector of FIG. 4.

FIG. 6A is a plan view of a jaw member of the end effector of FIG. 4 with an outer protective layer removed.

FIG. 6B is a plan view of a jaw member of the end effector of FIG. 4 with an outer protective layer and an electrode removed.

FIG. 7 is an exploded view of jaw members of the end effector of FIG. 4.

FIG. 8A is a perspective view of the jaw mechanism of the end effector of FIG. 4 in a first state of assembly with an actuation link.

FIG. 8B is a perspective view of the jaw mechanism of the end effector of FIG. 4 in a second state of assembly with the actuation link and with a clevis.

FIG. 9 is a block diagram of an embodiment of a teleoperable manipulator system.

### DETAILED DESCRIPTION

As noted above, it can be desirable to reduce the sizes of certain components of an instrument, such as a jaw mechanism, including its jaw members. For example, in certain circumstances, to reduce the overall size of a jaw mechanism, it may be desirable to reduce the size of the jaw members in a lateral dimension and/or in a height (thickness) dimension thereof. This may allow the jaw mechanism to be used in tighter spaces and may also allow the peripheral dimensions of the overall end effector to be reduced in some cases.

Reducing the dimensions of the jaw members can also give rise to some challenges. In particular, in some electrosurgical end effectors a jaw member of the jaw mechanism comprises an electrode for performing electrosurgical functions, with the electrode mounted on a jaw base. The jaw base provides the primary structural support of the jaw member and hence is usually configured to be relatively rigid and strong, whereas the electrode is configured for delivering electrosurgical energy to material (e.g., tissue) grasped between the jaw members and is generally relatively thin (in its height dimension) and less rigid. An insulating layer comprising an electrically insulating material (such as a plastic, ceramic, or other electrically insulating material, for example), is disposed between the jaw base and the electrode to prevent electrical shorting therebetween. An outer protective layer may also be disposed around a portion of the jaw member. If the overall end effector dimensions are reduced by reducing these various components of the jaw member in lateral and/or height dimensions, features thereof may become smaller and more intricate. For example, as the dimensions of the jaw base are reduced, features thereof become smaller and/or more intricate. This effect can be particularly pronounced near a distal end portion of the jaw member because the jaw member often tapers in a proximal to distal direction and thus the distal end portion of the jaw member may already have relatively small features compared to more proximal portions of the jaw member, resulting in these relatively small features becoming even smaller and more intricate upon reducing the overall dimensions of the jaw member. These smaller and more intricate features may be more difficult to manufacture in some cases. This is particularly true for the jaw base, as the materials and manufacturing techniques used to form the jaw base (e.g., machining of solid stainless steel or other similar material) can make it particularly difficult to form small and intricate features in the jaw base. Thus, attempting to reduce the dimensions of the jaw member by shrinking its components may make manufacturing more difficult.

Accordingly, some embodiments disclosed herein comprise electrosurgical end effectors with jaw members utilizing configurations that permit a relatively small height and/or lateral dimensions of the jaw member while retaining sufficient support and strength. The jaw member in accordance with some embodiments includes a jaw base supporting an electrode wherein the length of the jaw base (measured in the proximal-distal direction) terminates proximally of the distal end of the overall jaw member and supports an electrode extending distally beyond the distal end of the jaw base. Because the jaw base does not extend to the distal end of the jaw member, the distal end portion of the jaw member has fewer material layers, enabling a reduction in at least the height dimension of the distal end portion. Additionally, in some embodiments a lateral dimension of at least the distal end portion of the jaw member may also be reduced in addition to or in lieu of reducing the height dimension. Reducing height and/or lateral dimensions of the distal end portion of the jaw member by a jaw base configuration that does not extend to the distal end of the jaw member, according to embodiments disclosed herein, avoids the formation of small and intricate features in the distal end portion of jaw base. This is because the jaw base does not extend into the distal end portion of the jaw member, and thus reducing the dimensions of the distal end portion of the jaw member does not significantly affect the dimensions of the jaw base. Thus, in some embodiments disclosed herein, at least a distal end portion of the jaw member can be provided with reduced dimensions without significantly increasing the difficulty of manufacturing the jaw member.

Moreover, in some embodiments, in addition to reducing dimensions of the distal end portion of the jaw member, more proximal portions of the jaw member may also be provided with reduced height and/or lateral dimensions by reducing one or more dimensions of the proximal portions of components of the jaw member, including proximal portions of the jaw base. The more proximal portions of the jaw base tend to be relatively large as compared to the more distal portions thereof, for example due to tapering of the jaw member. Thus, the dimensions of the more proximal portions of the jaw base can be reduced without significantly increasing the difficulty of manufacturing the jaw base. In other words, reducing the dimensions of the more proximal portions of the jaw base does not generally result in features thereof becoming too small to be easily manufactured because those features are sufficiently large even with the reduced dimensions.

As noted above, the jaw base generally provides the primary structural support for the electrode, ensuring sufficient rigidity of the jaw member to resist flexing of the electrode during usage of the jaw mechanism. Although the electrode and insulating layer may generally contribute somewhat to the overall structural strength and rigidity of the jaw member, these layers are relatively thin and more flexible, and thus do not generally contribute as much structural support as the jaw base does. Thus, if the jaw base does not extend to the distal end of the jaw members as described above, and the electrode extends beyond the jaw base, this could result in the distal end portion of the electrode not having sufficient support and potentially flexing when used. Thus, in some embodiments disclosed herein, one or more support features may be added to the jaw member to provide increased structural support to the distal end portion of the electrode. In some embodiments, the support features are part of the electrode. For example, the support features may comprise a portion of the electrode that extends perpendicularly away from a contact surface of the electrode (i.e., a surface oriented to contact material grasped by the jaw) at least partially over a lateral face of the insulation layer at a position near the distal end portion of the electrode. For example, the electrode may comprise a flange at a lateral surface of the electrode, the flange extending perpendicularly from the contact surface and running along a length of the electrode, and support feature may comprise an enlarged portion of the flange that has a greater height dimension than other portion of the flange (i.e., the enlarged portion protrudes further from the contact surface than the remainder of the flange). In some embodiments, the flange (including the enlarged portion that forms the support feature) and the contact surface of the electrode may all be parts of the same unitary body, such as parts of the same piece of sheet metal that has been shaped (e.g., bent) to form the electrode.

In some embodiments, in addition to or in lieu of the support feature being part of the electrode, a support feature is part of the insulating layer. For example, a support feature may comprise a portion of the insulating layer that is thicker in a height dimension than other portions of the insulating layer, with the thicker portion extending distally beyond the distal end of the jaw base. In other words, the portion of the insulating layer that extends distally beyond the jaw base may be made thicker to increase the ability of the distal end portion of the insulating layer to provide structural support. In some embodiments, the overall thickness of the distal end portion of the jaw member can still be reduced despite the increase in thickness of the distal end portion of the insulating layer, due to the omission of the jaw base at this portion of the jaw member. In some embodiments, both of the aforementioned types of support features are used together, i.e., the electrode comprising a support feature extending perpendicularly from a contact surface thereof near a distal end portion thereof and the insulating layer having a thicker distal end portion. Thus, in embodiments disclosed herein, the supporting feature(s) may allow the distal end portion of the jaw member to be made sufficiently rigid despite the jaw base terminating prior to the distal end of the jaw member and a distal end portion of the electrode extending beyond the jaw base.

The addition of the aforementioned support features to the electrode and/or insulating layer could potentially make those individual components slightly more complex in structure and to manufacture. But, notwithstanding such potential increase in complexity of the electrode and/or the insulating layer, the overall level of difficulty in manufacturing of the jaw member may be reduced because the reductions in manufacturing difficulty accrued by omitting the jaw member at the distal end portion may outweigh any possible difficulties resulting from the addition of the support features to the electrode and/or insulating layer. Thus, in embodiments disclosed herein, the jaw member may have a relatively small overall dimensions, such as by reducing a height dimension and/or lateral dimensions at locations of the jaw member while maintaining sufficient strength and rigidity for the jaw member and also without significantly increasing the manufacturing difficult of the jaw member.

Another difficulty concerns the proximal coupling portions of the jaw members, which couple the jaw members together and to the rest of the end effector. It is generally difficult to provide jaw members whose proximal coupling portions allow for easy assembly of the jaw mechanism while also providing sufficient stability to the jaw members. Generally, a proximal connection portion of each the jaw member comprises one or more tangs that are coupled to a clevis, which in turn is coupled to a shaft of an instrument. The working portions of the jaw members that perform functions of the jaw member (such as, for example, grasping objects) extend distally from the tang(s). The jaw members are pivotally coupled together and to the clevis via pivot pin members engaged with apertures in the tangs and with apertures in the clevis. An actuation link is also engaged with the tangs and with the clevis so as to drive pivoting motion of the jaw members. For example, pin members of the actuation link are engaged with guide slots in the clevis and with ramped slots in tangs such that translation of the actuation link causes pivoting motion of the tangs and hence pivoting motion of the distal working portions of the jaw members. Some jaw members may have just one tang, which makes them relatively easy to assemble together and onto the actuation link, but this can also reduce the stability of the jaw members because there is only one contact region between the pivot pin member and each jaw member (i.e., at the aperture in the tang). Thus, such jaw member may be susceptible to lateral flexing and/or twisting of the jaw members, which may cause misalignment of the jaw members. This flexing or twisting may be combated by making tolerances of the apertures and pivot pin members stricter, but this may drive up manufacturing complexity and costs. In other jaw members, two spaced-apart tangs are provided, each with an aperture to engage a pivot pin member. As a result of each jaw member having two spaced apart apertures to engage the pivot pin members, these jaw members have improved stability to resist lateral flexing and twisting without needing as strict of tolerances. However, the two tangs can make it difficult to assemble the jaw members and the actuation link together while the actuation link is in an assembled state, as the tangs may interfere with the pin members of the actuation link and prevent insertion of the actuation link between the tangs. Thus, to facilitate assembly of the jaw members and actuation link, the actuation link may need to be in a disassembled state while being assembled with the jaw members (e.g., an axel that forms the pin members is separate from the rest of the actuation link during assembly), and then these separate parts of the actuation links may later be secured together (e.g., via welding) inside of the assembled jaw members. However, because the parts of the actuation link are located inside the assembled jaw member when they are being secured together, securing the parts together can be difficult.

Accordingly, various embodiments of jaw members disclosed herein have proximal connection portions (tangs) that facilitate assembly of the jaw mechanism and actuation link while also providing a robust connection at the pivot coupling of the jaw members. For example, in various embodiments, the pivot coupling between the jaw members provides multiple spaced-apart contact locations for each jaw member along the length of the pivot pin members. Such an arrangement, in which the load of the jaw members is distributed across more of lateral dimension (over more of the length of the pivot pin members) can resist offsetting or twisting of the jaw members, and also may reduce the need for strict tolerances that may otherwise be needed for pivot pin members and the apertures in the tang of a jaw member that receive them. Moreover, various embodiments having jaw members with proximal connection portions providing multiple spaced-apart contact locations with the pivot pin members also have configurations that enable the proximal connection portions (e.g., tangs) to be assembled with an actuation link that is already fully assembled, as opposed to configurations of such proximal connection portions in which portions of the actuation link are assembled after partial assembly of the jaw members on the actuation link or which make accessing the actuation link for assembly with the jaw members difficult.

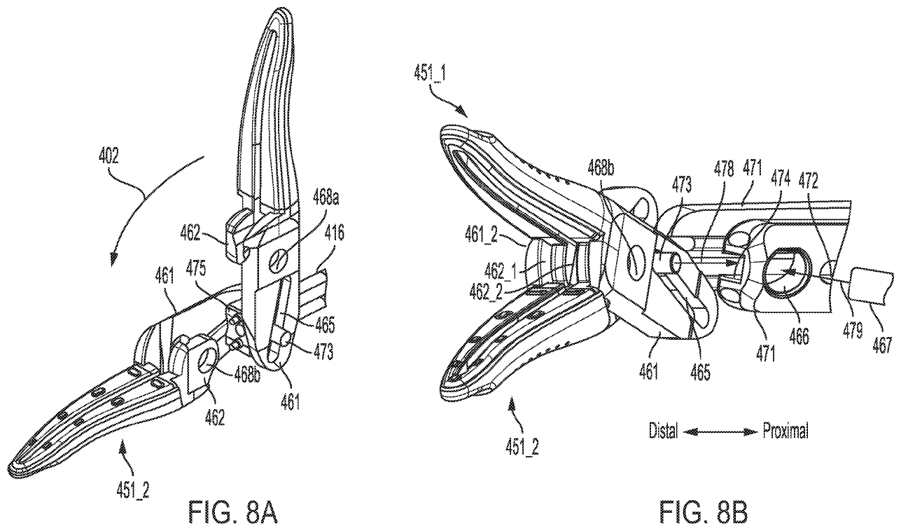

Accordingly, in some embodiments, the proximal connection portion of each jaw member comprises two tangs positioned on opposite sides of a longitudinal centerline of the jaw member and each of the tangs has an aperture to receive a pivot pin member so as to provide multiple points of contact with the pivot pin members as described above, which results in increased stability to resist lateral offset and twisting, as described above. Moreover, to facilitate assembly of the jaw members with the actuation link, one of the tangs of each jaw member is shorter than the other tang of the jaw member—i.e., each jaw member comprises a long tang and a short tang. The long tang of each jaw member comprises the above-described ramped slot to engage with a pin member of the actuation link. However, the short tang does not comprise a ramped slot, and instead terminates at a location distal of the ramped slot portion of the long tang. This arrangement allows jaw members to be assembled onto the actuation link by positioning the jaw members on opposite sides of the fully assembled actuation link and then moving the jaw members laterally toward one another, with the ramped slots of the long tangs receiving the pin members of the actuation link as the two jaw members come together. This assembly process is enabled because the short tangs of the jaw members can be positioned during the assembly process such that they do not collide with one another or with the actuation link as the two jaw members are brought together onto the actuation link. Once the jaw members are assembled onto the actuation link, the jaw members can be coupled to the clevis by inserting pivot pin members through apertures in the clevis and the apertures in each of the long and short tangs of each jaw member. An embodiment of assembling the jaw mechanism is described in greater detail below with reference to FIGS. 8A and 8B.

Turning now to the figures, various embodiments are described below in greater detail.

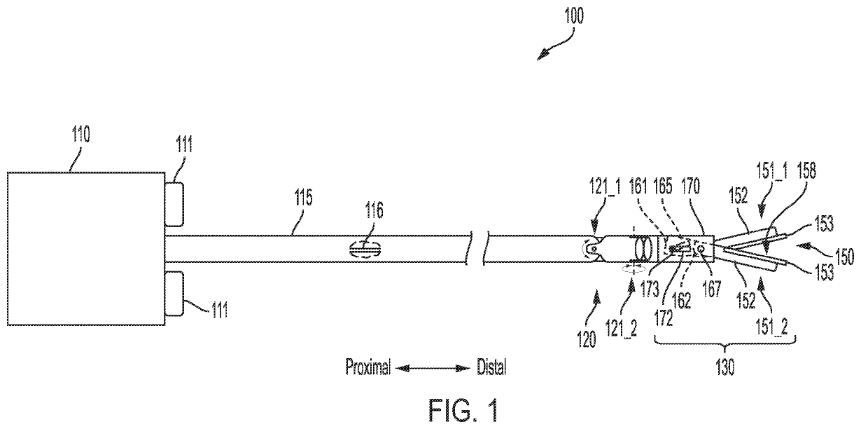

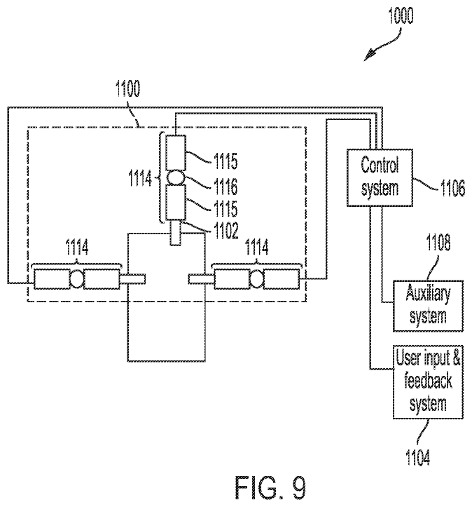

FIG. 1 is a schematic diagram illustrating a side view of an embodiment of an instrument 100. In some embodiments, the instrument 100 may be used and controlled via a computer-controlled system, such as a system 1000 described with reference to FIG. 9, discussed further below. In other embodiments, the instrument 100 may a manually operable instrument.

As shown in FIG. 1, the instrument 100 comprises an end effector 130, a transmission assembly 110, and a shaft 115 coupled to and extending between the transmission assembly 110 and the end effector 130. In some embodiments, the end effector 130 is disposed at a distal end portion of the instrument 100 and the transmission assembly is disposed at a proximal end portion of the instrument 100 (proximal and distal directions referenced herein are as illustrated in FIG. 1). The instrument 100 may also comprise one or more articulable structures 120 along the shaft 115 at one or more locations between the end effector 130 and the transmission assembly 110. In some embodiments, an articulable structure 120 couples the end effector 130 to the shaft 115. In some embodiments, an articulable structure 120 couples one portion of the shaft 115 to another portion of the shaft 115. An articulable structure 120 may comprise, for example, any component that couples two parts together in a manner that allows for relative motion between the parts, such as one or more joints 121 (e.g., the joints 121_1 and 121_2 shown in FIG. 1), flexible sections (e.g., via material properties of the shaft and/or relief features in the shaft, and other mechanisms to permit elastic flexing/bending of the shaft). The articulable structures 120 (when present) provide one or more degrees of freedom of motion of the instrument 100, such as for moving the end effector 130 as a whole relative to the shaft so as to orient it as desired relative to a workspace. In some embodiments, at least one articulable structure 120 comprises a wrist comprising multiple joints 121 coupled directly or indirectly together to provide multiple degrees of freedom of motion of the end effector 130 relative to the shaft 115, such as pitch, yaw, roll, or any combination thereof.

The transmission assembly 110 comprises one or more drive inputs 111 configured to receive driving forces and/or other inputs that control functions of the instrument 100, such as movements of the instrument 100 (including, e.g., movements of the shaft 115, the end effector 130, and/or an articulable structure 120) and/or functions of the end effector 130. The drive inputs 111 may be arranged to interface with drive outputs of a manipulator system, as described further below with reference to FIG. 9, or they may be driven by manual manipulation such as via various inputs at the transmission assembly 110 itself. Examples of drive inputs 111 include, but are not limited to, rotational couplers (discs), levers, linear motion inputs/outputs, gears, capstans, and pulleys. The driving forces may be transferred from the transmission assembly 110 to the end effector 130 and articulable structures 120 (if present) via actuation elements 116 extending through the shaft 115. Actuation elements 116 can take a variety of forms, such as cables, wires, filaments, rods, rigid tubes, bars, plates, push-coils, etc., or combinations thereof. In some embodiments, conduits may also extend through the shaft 115 to deliver various other drive inputs, such as electrical conduits for delivering electrical energy and/or fluidic conduits to deliver fluids, pressure, and/or suction to the end effector 130.

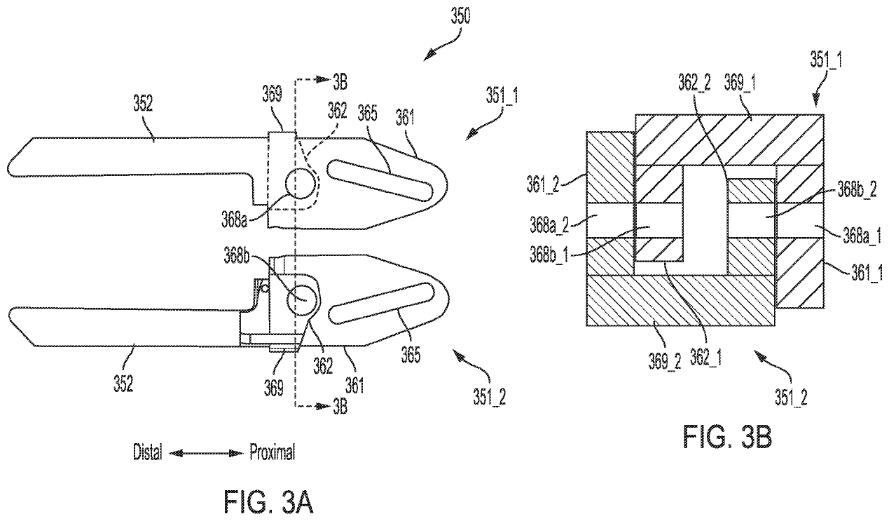

The end effector 130 comprises a jaw mechanism 150 coupled to a clevis 170, which in turn is coupled to the shaft 115 directly or via an articulable structure 120 as shown in FIG. 1. The jaw mechanism 150 comprises two jaw members 151 (e.g., jaw member 151_1 and 151_2) which are each coupled to the clevis 170 by a pivot pin member 167 engaged with an aperture in each jaw member 151, such that the jaw members 151 are movable relative to each other (e.g., by pivoting) between open and closed states. In particular, as the jaw mechanism 150 moves between open and closed configurations, the distal working portions of the jaw members 151 move towards and away from one another. In the closed configuration, the distal working portions of the jaw members 151 are positioned approximately parallel to one another such that respective opposing contact surfaces 158 of the jaw members 151, are positioned close to and facing one another (or in contact with one another). Thus, if the jaw mechanism 150 is brought into the closed configuration while an object is positioned between the jaw members 151, the respective contact surfaces 158 of the jaw members 151 come into contact with opposite sides of the object, thus grasping the object. The above-described pivoting motion of the distal working portions of the jaw members 151 is driven by movement of proximal connection portions of the jaw members 151, which comprise at least one tang (for example, a long tang 161 and a short tang 162, in some embodiments, as described in greater detail below). The tangs of the jaw members 151 comprise the above-noted apertures (see, e.g., apertures 368a and 368b in FIGS. 3A and 3B) that receive and engage with the pivot pin members 167 to couple the jaw members 151 to the clevis 170. One pivot pin member 167 is positioned on a first lateral side of the end effector 130 to engage with a part of the clevis and those of the tang(s) that are located on the first side, and another pivot pin member 167 is positioned on a second lateral side of the end effector 130, opposite from the first lateral side, so as to engage with another part of the clevis and the other tang(s) that are located on the second side. In some embodiments, the aforementioned two pivot pin members 167 are separate and distinct bodies, such as two separate pins. In other embodiments, the two pivot pin members 167 are part of the same body; for example, the two pivot pin members 167 may correspond to opposite end portions of a single pivot pin, with the single pivot pin extending through all of the above-noted apertures. In addition, at least one of the tangs (e.g., the long tang 161, in some embodiments) of each jaw member 151 comprises a ramped slot 165, which is engaged with one of a pair of pins members 173 of an actuation link. The actuation link comprises a main body and the two pin members 173 coupled to and extending laterally from the main body in opposite directions (note that the two pin members 173 could be two separate bodies, such as two separate pins coupled to the main body, or the two pin members 173 could be two parts of the same single body, such as two end portions of an axel coupled to the main body). The pin members 173 of the actuation link are further engaged with guide slots 172 in the clevis 170, which constrain motion of the pin members 173 to only translations along the guide slots 172. As the pin members 173 translate along the guide slots 172, they interact with the ramped slots 165 to force the tangs to move, thus causing the distal working portions of the jaw members 151 to pivot. The actuation link to which the pin members 173 are coupled is itself coupled to an actuation element 116, which is actuatable to drive translation of the actuation link and thus opening and closing of the jaw mechanism 150.

In some embodiments, the end effector 130 also comprises a movable component (not illustrated in FIG. 1) which is coupled to another actuation element 116 and movable (e.g., translatable) relative to the jaw members 151. For example, the movable component may be a cutting element to cut material grasped between the jaw members 151 or a staple firing shuttle.

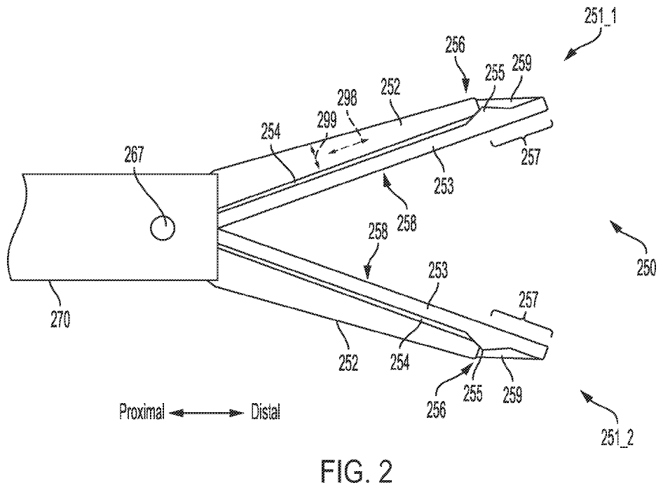

In some embodiments, one or both of the jaw members 151 comprises an electrode 153 for delivering electrical energy to the material grasped between the jaw members 151. The electrode 153 comprises a conductive material (e.g., stainless steel, brass, copper, or other suitable electrically conductive materials) disposed on a jaw base 152, with the jaw base 152 providing structural support and rigidity to the jaw member 151. For example, in some embodiments, the electrode 153 may be formed from sheet metal that has been worked (e.g., cut, stamped, bent, or otherwise formed) into the desired shape. An insulating layer (not shown in FIG. 1) formed from an electrically insulating material (e.g., a plastic, ceramic, or other suitable electrically insulating material) may be disposed between the electrode 153 and the jaw base 152. An exposed surface of the electrode 153 (see, e.g., surface 258 in FIG. 2) is arranged so as to face the opposite jaw member 151, and this surface of the electrode 153 forms a contact surface 158 of the jaw member 151 that will contact an object when the object is grasped between the jaw members 151. In such embodiments, electrically conductive conduits, such as wires or cables, for example, extend through the shaft 115 to the electrodes 153 to electrically couple the electrode 153 to an electrical power source, such as an electrosurgical unit (ESU), which power source can be coupled to terminals at the transmission assembly 110. In other embodiments of the jaw mechanism 150, the electrode 153 is omitted from one or both of the jaw members 151 and the instrument may be an instrument that is not configured to deliver electrosurgical energy, but one configured to perform various grasping functions, such as a stapler, forceps, and other similar instruments.

In some embodiments in which the jaw members 151 include electrodes 153, the end effector 130 may be configured as an electrosurgical instrument (e.g., a vessel sealing instrument), with electrodes 153 being configured to deliver electrosurgical energy (e.g., bipolar electrosurgical energy and/or monopolar electrosurgical energy) to perform electrosurgical functions such as sealing and/or cutting tissue (e.g., a blood vessel) grasped between the jaw members 151. In some of these embodiments, the jaw mechanism 150 also comprises a mechanical cutting element (not shown) that translates relative to the jaw members 151 along a proximal-distal direction to cut tissue grasped between the jaw members 151.

In some embodiments in which a jaw member 151 comprises an electrode 153, the jaw base 152 terminates proximally of a distal end of the jaw member 151. In other words, in these embodiments, the electrode 153 extends distally beyond the jaw base 152. This may allow at least a distal portion of the jaw member 151 to be relatively thin (e.g., compared to conventional jaw members of similar shape) in a lateral and/or height dimension (the lateral and height dimensions being perpendicular to a longitudinal dimension of the jaw member 151). Moreover, in these embodiments, a support feature (not visible in FIG. 1) is provided on the electrode 153, the insulating layer, or both to provide increased structural support to a distal end portion of the electrode 153, which extends beyond the end of the jaw base 152, to compensate for the omission of the jaw base 152 in this region. In some embodiments, both the electrode 153 and the insulating layer are provided with a support feature, while in other embodiments just one or the other is provided with a support feature. Embodiments of such support features are described in greater detail below with reference to FIG. 2.

FIG. 2 illustrates a jaw mechanism 250 that can be used as one embodiment of the jaw mechanism 150 in which an electrode extends distally beyond the jaw base. Similar components of the jaw mechanisms 150 and 250 are given reference numbers with the same two right-most digits, such as 151 and 251. As shown in FIG. 2, the jaw mechanism 250 comprises two jaw members 251 (i.e., 251_1 and 251_2) pivotably coupled to a clevis 270 by a pivot pin member 267. The jaw members 251 each comprise a jaw base 252, an insulating layer 254 disposed on the jaw base 252, and an electrode 253 disposed on the insulating layer 254. As shown in FIG. 2, the jaw base 252 terminates proximally of the distal end of the jaw member 251. In other words, a distal portion 257 of the electrode 253 extends distally beyond the distal end 256 of the jaw base 252. Moreover, the electrode 253 comprises a support feature 255 extending perpendicularly from a contact surface 258 of the electrode 253 (the contact surface 258 being generally perpendicular to a height dimension 299 of the jaw member 251 and arranged to contact an object grasped by the jaw mechanism 250). In other words, the support feature 255 extends from the contact surface 258 in a direction generally parallel to a height dimension 299 of the jaw member. For example, the support feature 255 may have an apex that is located further from the contact surface of the electrode 253 along a direction parallel to the height dimension 299 than any other portion of the electrode 253. In some embodiments, the support feature 255 is located near the distal end 256 of the jaw base 252 so as to provide increased structural strength at the distal end portion 257 of the electrode 253, and in some embodiments at least a portion of the support feature 255 extends distally beyond the distal end 256 of the jaw base 252. In particular, in some embodiments the apex of the support feature 255 is located near the distal end 256 of the jaw base 252 and a sloped edge of the support features 255 extends distally beyond the distal end 256 of the jaw base 252.

In addition, as illustrated the embodiment of FIG. 2, the insulating layer 254 optionally also comprises a support feature in the form of a thickened portion 259, which is thicker in the height dimension 299 than other portions of the insulating layer 254. As shown in FIG. 2, the thickened portion 259 comprises the portion of the insulating layer 254 that extends distally beyond the distal end 256 of the jaw base 252. The support feature 255 of the electrode 253 and the thickened portion 259 of the insulating layer 254 together provide increased support and rigidity to the distal end portion 257 of the electrode 253 to compensate for the lack of the jaw base 252 in this region, thus reducing the likelihood of the electrode 253 bending when in use. Moreover, because the jaw base 252 does not extend to the distal end of the jaw member 251, at least the distal working portion of the jaw member 251 can be made thinner in a height and/or lateral dimension without requiring the formation of small and intricate features in the distal end of the jaw base 252, and thus reducing the thickness of the jaw member 251 does not significantly increase the difficulty of manufacturing the jaw member 251.

In the embodiment of FIG. 2 described above, both the electrode 253 and the insulating layer 254 comprise support features (e.g., the support feature 255 and thickened portion 259) to support the distal portion 257 of the electrode 253. However, in some embodiments of the jaw mechanism 150 the electrode 153 comprises such a support feature while the insulating layer does not, and in other embodiments the insulating layer comprises the support feature and the electrode 153 does not.

Returning now to FIG. 1, in some embodiments of the jaw mechanism 150, the proximal connection portions of each jaw member 151 comprises a long tang 161 and a short tang 162. The long and short tangs 161 and 162 are positioned on opposite sides of a longitudinal centerline of the jaw member 151 and each of tangs 161 and 162 has an aperture to receive the pivot pin member 167, thus providing two spaced-apart points of contact on the proximal connection portions of each jaw member 151 for the pivot pin member 167, which increases stability of the jaw member 151 to resist lateral offsetting or twisting, as described above. The long tang 161 of each jaw member 151 also comprises a ramped slot 165 to engage with a pin member 173 of the actuation link, whereas the short tang 162 does not comprise a ramped slot and instead terminates at approximately where the ramped slot portion of the long tang 161 begins. This configuration allows the jaw members 151 to be assembled together on the actuation link in a fully assembled state of the actuation link (i.e., a state in which the pin members 173 are assembled with/coupled to a remainder of the actuation link), as will be described in greater detail below with reference to FIGS. 3A and 3B.

FIGS. 3A and 3B illustrate a jaw mechanism 350 that can be used as one embodiment of the jaw mechanism 150 in which jaw members comprise long and short tangs. Similar components of the jaw mechanisms 150 and 350 are given reference numbers with the same two right-most digits, such as 351 and 351. FIG. 3A illustrates a pair of jaw members 351 in an exploded view with the jaw member 351_1 above the jaw member 351_2, and FIG. 3B comprises a cross-section illustrating the jaw members 351_1 and 351_2 in a partially assembled state. As shown in FIG. 3, a distal working portion of each jaw member 351 compromises a grasping portion 352, and a proximal connection portion of each jaw member 351 comprises a long tang 361 and a short tang 362. The grasping portion 352 of the jaw member 351 may comprise a jaw base (e.g., the jaw base 152 described above), as well as additional parts configured to perform various operations, including at least grasping an object between the jaw members 351 when closed. The grasping portion 352 thus has a contact surface that is to contact the grasped object. The grasping portion 352 may also be configured to perform other operations as well in some embodiments, such as electrosurgical operations, stapling, cutting, with the grasping portion 352 optionally comprising other components for performing such functions as would be understood by one of ordinary skill in the art. The grasping portion 352 is coupled to the long and short tangs 361 and 362 by an intermediate section 369.

Each of the long and short tangs 361 and 362 comprises apertures 368a and 368b, respectively, arranged to receive pivot pin members (not illustrated) to pivotable couple the jaw members 351_1 and 351_2 together and to a clevis (such as the clevis 170). In particular as shown in FIG. 3B, when the jaw members 351_1 and 351_2 are assembled, the aperture 368a_2 of the long tang 361_2 of the jaw member 351_2 is aligned with the aperture 368b_1 of the short tang 362_1 of the jaw member 351_1 so that a first pivot pin member (not illustrated) can be inserted through these apertures 368a and 368b and through a first aperture in one side of the clevis. Similarly, in this state the aperture 368a_1 of the long tang 361_1 of the jaw member 351_1 is aligned with the aperture 368b_2 of the short tang 362_2 of the jaw member 351_2 so that a second pivot pin member (not illustrated) can be inserted through these apertures 368a and 368b and through a second aperture in a second side of the clevis. (As noted above, the pivot pin members may be two separate bodies or may be two parts of a single body.) Moreover, as shown in FIG. 3B, in the assembled state the tangs 361 and 362 of the two jaw members 351_1 and 351_2 are interleaved, with the long tang 361 of one jaw member 351 positioned adjacent to the short tang 362 of the other jaw member 351. In FIG. 3B, the short tangs 362_1 and 362_2 are positioned inside of the long tangs 361_1 and 361_2, but in other embodiments the reverse arrangement is used.

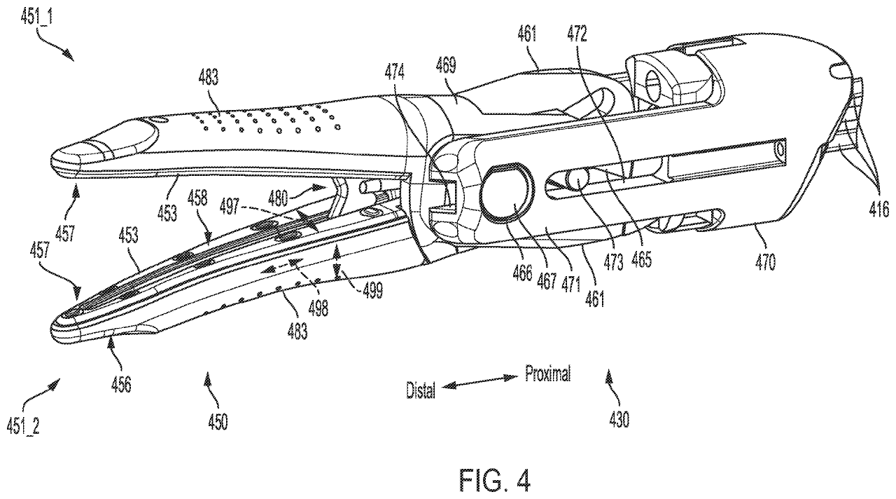

As shown in FIG. 3A, the long tang 361 of each jaw member comprises a ramped slot 365, which is configured to receive a pin member of an actuation link (not illustrated). In the assembled state of the jaw member 351, the long tangs 361 of the two jaw members 351 are positioned opposite from one another on either side of a longitudinal centerline of the jaw mechanism 350, with the actuation link being positioned between the long tangs 361 such that a pin member on one side of the actuation link engages the ramped slot 365 of one of the long tangs 361 and a second pin member on the opposite side of the actuation link engages the ramped slot 365 of the other long tang 361. The pin members of the actuation link also engage guide slots arranged on opposite sides of the clevis (not shown in FIGS. 3A and 3B) so as to constrain motion of the actuation link to translation along the proximal-distal direction, as explained in greater detail below with reference to FIG. 4. The ramped slot 365 is angled relative to the longitudinal dimension of the jaw member 351 such that as the actuation link is driven to translate, the pin members of the actuation link push against the walls of the ramped slots 365 and force the jaws to pivot about the pivot pin members.

As shown in FIG. 3A, in some embodiments the short tang 362 of each jaw member 351 does not comprise a ramped slot 365. Instead, the short tang 362 of each jaw member 351 terminates approximately at or distally of where the ramped slot 365 of the long tang 361 begins. This configuration of the short tang 362 allows the jaw members 351_1 and 351_2 to be assembled onto the actuation link by positioning the jaw members 351_1 and 351_2 on opposite sides of the fully assembled actuation link, orienting the jaw members 351_1 and 351_2 at an angle relative to one another (e.g., in an opened configuration of the jaw mechanism) with the ramped slots 365 being aligned with the pin members of the actuation link, and then moving the jaw members 351_1 and 351_2 laterally towards one another until the ramped slots 365 of the long tangs 361 move onto the pin members of the actuation link. Because of the length of the short tangs 362, the respective short tangs 362 of the jaw member 351_1 and 351_2 can move past one another without collision as the two jaw members are moved together in the above-described process. This assembly process allows the actuation link to be fully assembled outside of the jaw members 351, which can greatly ease the manufacturing process. An embodiment of the above-described process is described in greater detail below in the context of a particular embodiment with reference to FIGS. 8A and 8B

In some embodiments, an electrode is disposed on the grasping portion 352, such as the electrodes 153 and 253 described above. In other embodiments, no electrode is present. In particular, the above-described long and short tangs 361 and 362 can be used with any type of jaw mechanism of any type of end effector, including but not limited to an electrosurgical end effector, a stapler, or forceps, for example.

Returning to FIG. 1, although the jaw members 151 are illustrated as having both the electrode 153 and the long and short tangs 161 and 162, in some embodiments the jaw members 151 do not necessarily include both of these aspects together. More specifically, in some embodiments one or both jaw members 151 comprise both the electrode 153 and the long/short tangs 161 and 162, in other embodiments one or both jaw members 151 comprise the electrode 153 but not the long and short tangs 161 and 162, and in still other embodiments one or both jaw members 151 comprise the long and short tangs 161 and 162 but not the electrode 153. Moreover, as noted above, in some embodiments in which the electrode 153 is present, the electrode 153 extends distally beyond the distal end of the jaw base 152 and a support feature is provided in the electrode 153 and/or insulating layer, but in other embodiments of the jaw mechanism 150 one or more jaw members 151 has the electrode 153 but the electrode 153 does not extend significantly beyond the distal end of the jaw base 152 and thus no additional support features are provided.

Turning now to FIGS. 4-8B, an embodiment of an end effector 430 is described in greater detail. The end effector 430 may be used as the end effector 130 described above. Some components of the end effector 430 may be used as components of the end effector 130 described above, and thus the descriptions of the components of the end effector 130 above are applicable to the related components of the end effector 430. These related components are given reference numbers having the same right-most two digits, such as 150 and 450. Although the end effector 430 is one embodiment of the end effector 130, the end effector 130 is not limited to the end effector 430. As elements of the end effector 430 are described, one or a few figures which are thought to be particularly pertinent to the aspect will be noted, but it should be understood that other figures besides those that are identified may also illustrate the same part from other perspectives. Thus, the description below will not necessarily describe the figures FIGS. 4-8B separately and in strict sequence.

As shown in the embodiment of FIG. 4, the end effector 430 comprises a clevis 470 and a jaw mechanism 450 coupled to the clevis 470. The clevis 470 comprises two arms 471 arranged on opposite side of a longitudinal centerline of the end effector 430, and the jaw mechanism comprise two jaw members 451 (e.g., 451_1 and 451_2) arranged between and pivotably coupled to the arms 471. The clevis 470 is in turn coupled to an instrument shaft (such as shaft 115) directly or via an articulable structure (such as articulable structure 120). The end effector 430 further comprises a cutting element 480 as shown in FIG. 4 and an actuation link 475 as shown in FIG. 8A (a pin member 473 of the actuation link 475 is visible in FIG. 4).

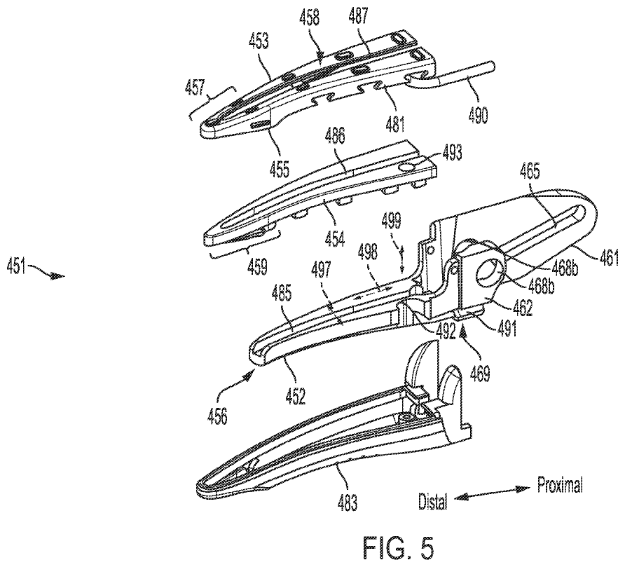

As shown in FIGS. 5-7, a proximal connection portion of each jaw member 451 comprises a long tang 461 and a short tang 462 which are pivotably coupled to the clevis 470 by one or more pivot pin members 467 (the long and short tangs 461 and 462 are described in greater detail below). The pivot pin members 467 engage (i.e., are received within) apertures 468a and 468b in the long and short tangs 461 and 462 of the jaw members 451 and also engage apertures 466 in the arms 471 of the clevis 470, thereby pivotably coupling the jaw members 451 to the clevis 470. One pivot pin member 467 engages one arm 471 and another pivot pin member 467 engages the other arm 471. In some embodiments, these pivot pin members 467 are two separate bodies, such as two distinct pivot pins. In other embodiments, the pivot pin members 467 are two parts of a single body, such as a single pivot pin that extends through all of the aforementioned apertures 466, 468a, and 468b. Regardless of whether the pivot pin members 467 are formed as two separate bodies or two parts of one single body, the apertures 468a and 468b of each jaw member 451 provide the jaw member 451 with two spaced-apart points of contact for engaging with the pivot pin members 467, thus providing stability to resist twisting without requiring extremely high tolerances between the aperture and pivot pin members 467.

Moreover, as shown in FIGS. 5-8B, the long tang 461 of each jaw member 451 comprises a ramped slot 465 to engage with a pin member 473 of the actuation link 475. Note that the pin members 473 of the actuation link 475 may be two separate parts or they may be two parts of the same body, such as two opposite ends of an axel, which is coupled to a main body of the actuation link 475 such that the ends of the axel protrude from opposite sides of the main body to form the pin members 473. In an assembled state of the actuation link 475, the axel is secured to (e.g., via welding, adhesive, mechanical fasteners, etc.) the actuation link 475. As shown in FIG. 4, the pin members 473 also engage guide slots 472 in the arms 471 of the clevis 470, which constrains motion of the actuation link 475 to only translation along the proximal-distal direction. As the actuation link 475 translates, the pin members 473 slide within the guide slots 472 and push against the walls of the ramped slots 465, thus forcing the jaw members 451 to pivot about the pivot pin members 467. Thus, the jaw mechanism 450 can be opened and closed by driving the actuation link 475 to translate along the proximal-distal direction.

As shown in FIGS. 4 and 8A, actuation elements 416 extend along the instrument shaft (not shown) and into and/or through the clevis 470. One or more of the actuation elements 416 are coupled with the actuation link 475 (in FIG. 8A, two actuation elements 416 are coupled to the actuation link 475, but in other embodiments one actuation element 416 is used), and are actuatable to drive the translation of the actuation link 475 relative to the clevis 470. An additional actuation element 416 is coupled to the cutting element 480 to drive translation thereof as those of ordinary skill in the art are familiar with.

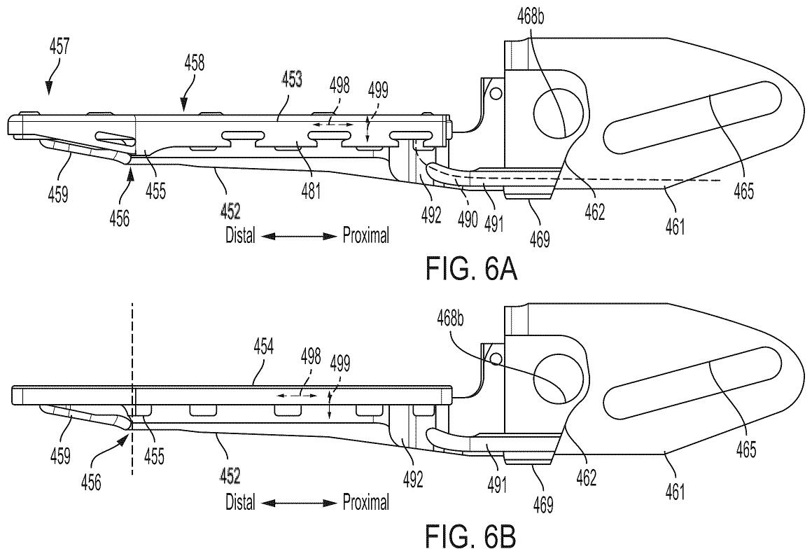

With reference to FIG. 5, in addition to the long and short tangs 461 and 462 mentioned above, each jaw member 451 also comprises a jaw base 452, an insulating layer 454 disposed on the jaw base 452, and an electrode 453 disposed on the insulating layer 454. As shown in FIGS. 5-6B, the jaw base 452 is coupled to and extends distally from the long and short tangs 461 and 462. Moreover, an intermediate portion 469 may also be coupled to the jaw base 452 and to the long and short tangs 461 and 462. The jaw base 452 may be formed from a relatively strong and rigid material, such as stainless steel, and may provide the primary structural support for the jaw member. As noted above, the jaw members 451 also comprise an electrode 453, which may be formed from an electrically conductive material, such as from sheet metal. The electrode 453 may be used as the electrode 153 described above. The electrode 453 comprises a contact surface 458, which is approximately perpendicular to a height dimension 499 of the jaw member 451 and arranged so as to face the opposite jaw member 451 such that the surface 458 can contact an object when the object is grasped between the jaw members 451. As shown in FIGS. 5 and 6A, the electrode 453 also comprises a flange 481, which extends perpendicularly from the contact surface 458 (i.e., along a height dimension 499) and extends along a length of the electrode adjacent to (partially covering) a lateral face of the insulating layer. The flange 481 may aid in attaching the electrode 453 to the other components of the jaw member 451, and may also provide some structural support.

As shown in FIGS. 5 and 6A, a distal portion 457 of the electrode 453 extends distally beyond a distal end 456 of the jaw base 452. In other words, the jaw base 452 terminates proximally of the distal end of the jaw member 451. Similarly, a distal portion 459 of the insulating layer 454 extends distally beyond the distal end 456 of the jaw base 452, with the distal portion 459 of the insulating layer 454 being disposed under the distal portion 457 of the electrode 453. As shown in FIGS. 5 and 6A, the electrode 453 comprises a support feature 455 which extends perpendicularly from the contact surface 458. In other words, the support feature 455 extends from the contact surface 458 in a direction generally parallel to a height dimension 499 of the jaw member 451. The support feature 455 may be a part of the flange 481, but may have a greater height profile than a remainder of the flange 481. As show in FIG. 6A, the support feature 455 is roughly triangular in shape and comprises an apex connected to two sloped edges. Moving from proximal to distal, one sloped edge of the support feature 455 begins at the flange 481 and extends at an angle distally and away from the contact surface 458 until reaching the apex and then from the apex the other sloped edge extends at an angle distally and toward the contact surface 458. In other words, moving distally from the apex, the support feature 455 tapers (in the height dimension) towards the distal end of the electrode 453, and moving proximally from the apex the support feature 455 tapers (in the heigh dimension) towards the flange 481. The apex of the support feature 455 is located adjacent to the distal end 456 of the jaw base 452, with a portion of the support feature 455 extending distally beyond the distal end 456 of the jaw base 452. With this configuration, the support feature 455 can provide increased support to the distal portion 457 of the electrode 453 to help prevent flexing of the electrode 453 when the jaw mechanism 450 grasps an object. While the flange 481 may also provide some support, it may not be sufficient in some circumstances. However, the greater height of the support feature 455 in the height dimension 499, as compared to the remainder of the flange 481, together with the geometry of the support feature 445, may allow the support features 455 to provide increased support to the distal portion 457 of the electrode 453.

In addition, as shown in FIG. 6B, in which the electrode 453 is removed to better show the insulating layer, the insulating layer 454 also comprises a support feature in the form of a distal portion 459 that is relatively thick in the height dimension 499 as compared to other portions of the insulating layer 454. The thickened distal portion 459 comprises a portion of the insulating layer that extends beyond the distal end 456 of the jaw base 452. This thickened distal portion 459 of the insulating layer 454 contributes some additional structural support to the distal portion 457 of the electrode 453, which together with the increased support provided by the support feature 455 may be sufficient to mitigate the omission of the jaw base 452 under the distal portion 457. Moreover, because the jaw base 452 does not extend to the distal end of the jaw member 451, at least the distal portion of the jaw member 451 can be made thinner in a height dimension 499 and/or a lateral dimension 497 without requiring the formation of small and intricate features in the distal end of the jaw base 452. Thus, the reduction in thickness of the jaw member 451 does not significantly increase the difficulty of manufacturing the jaw member 451. Other portions of the jaw member 451, in addition to the distal end portion, may also be reduced in the height dimension 499 and/or a lateral dimension 497.

As shown in FIG. 5, an electrical conduit 490 (e.g., a wire or a cable) is coupled to the electrode 453. As shown in FIG. 6A, which indicates a path of the electrical conduit 490 via dashed lines, the electrical conduit 490 may extend from the electrode 453 in a first direction aligned with a height dimension 499 through an opening 493 in the insulating layer 454 (see FIG. 5) and through a first groove 492 in the jaw base 452 (see FIGS. 5 and 6A), and then may turn to extend in a second direction aligned with longitudinal dimension 498 through a second groove 491 in the jaw base 452 (see FIGS. 5 and 6A). After passing through the second groove 491, the electrical conduit 490 may extend proximally through the clevis 470 to the shaft and then ultimately to an electrical power source configured to supply electrical energy to the electrode 453.

As shown in FIGS. 4 and 5, the jaw member 451 may also comprise an outer protective layer 483, which may cover portions of the jaw base 452 and insulating layer 454, as well as over some lateral portions of the electrode 453. A contact surface 458 of the electrode 453 is not covered by the outer protective layer 483. In some embodiments, the outer protective layer 483 is over-molded over the other portions of the jaw member 451.

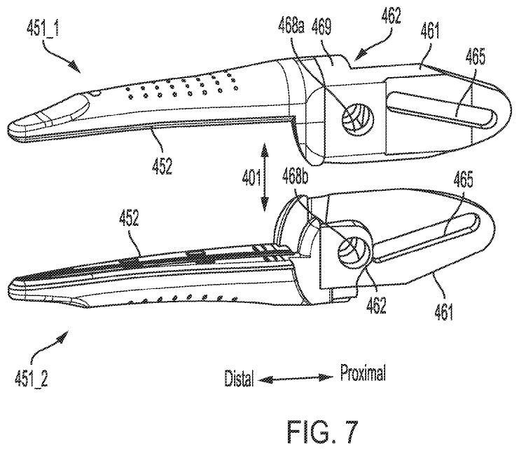

Turning now to FIGS. 6A-8B, the long and short tangs 461 and 462 of the jaw member 451 and an embodiment of a process of assembling the jaw mechanism 450 will now be described in greater detail with reference to FIGS. 6A-8B. The long and short tangs 461 and 462 are positioned on opposite sides of a longitudinal centerline of the jaw member 451. The two jaw members 451_1 and 451_2 may be positioned such that their long and short tangs 461 and 462 arranged on opposite sides as one another when assembled together, as shown in the exploded view of FIG. 7. When the jaw members 451_1 and 451_2 are brought together, their respective long and short tangs 461 and 462 interleave one another, as shown in FIG. 8B. In each jaw member 451, the long tang 461 comprises the ramped slot 465, whereas the short tang 462 does not comprise a ramped slot and instead ends distal of where the ramped slot is on the long tang. This configuration allows the jaw members 451 to be assembled together and onto the actuation link 475 in a fully assembled state of the actuation link 475, for example by the following process.

First, with reference to FIG. 8A, the jaw members 451_1 and 451_2 are positioned on opposite sides of the fully assembled actuation link 475. The fully assembled actuation link 475 is in a state in which the pin members 473 are formed in and coupled to a main body of the actuation link 475, such as by coupling an axel to the main body as described above, and a state in which the main body of the actuation link 475 is coupled to the actuation element 416. For example, the main body may be welded or mechanically fastened (e.g., crimped) to the actuation element(s) 416. The assembly of the actuation link 475 occurs while the actuation link 475 is outside of the jaw members 451, thus facilitating access to and easing the assembly of the actuation link 475. The jaw members 451_1 and 451_2 are arranged such that they are at an angle relative to one another while also having their respective ramped slots 465 aligned with the pin members 473 of the actuation link 475. The jaw members 451_1 and 451_2 are then moved laterally towards one another and towards the actuation link 475 until the respective ramped slots 465 of the jaw members 451_1 and 451_2 have engaged with the pin members 473 of the actuation link 475, as shown in FIG. 8A. As shown in FIG. 8A, because of the relatively short length of the short tangs 462 and because the jaw members 451_1 and 451_2 are oriented at an angle relative to one another, the short tangs 462 of the jaw members 451_1 and 451_2 do not collide with one another or with the actuation link 475 as the jaw members 451_1 and 451_2 are moved laterally towards one another. Thereafter, the jaw members 451 are pivoted in a direction (402 in FIG. 8A) that brings their distal ends closer together until all of the apertures 468a and 468b are aligned with one another, and the pin members 473 (still engaged with the jaw members 451) may be further engaged with a track 474 in an interior wall of the arms 471, as shown in FIG. 8B. This track 474 leads to the guide slot 472, and the jaw members 451 and the actuation link 475 may be moved proximally relative to the clevis 470 such that the pin members 473 are brought into the guide slot 472 and the apertures 468a and 468b of the jaw members 451 are aligned with the apertures 466 in the arms 471. With the apertures 468a, 468b, and 466 all aligned, the pivot pin members 467 may be inserted therethrough as indicated by arrow 479 in FIG. 8B and secured to the clevis 460 (e.g., via welding, mechanical fasteners, adhesives, etc.), thus attaching the jaw mechanism 450 to the clevis 460. While the pivoting of the jaw member 451 and the engagement of the pin members 473 with the track 474 are described above in a certain sequence, those having ordinary skill in the art would appreciate these could be performed in other orders, such as engaging the pin members 473 with the track 474 first and then pivoting the jaw members 451 thereafter. The assembly process described above allows the actuation link 475 to be fully assembled outside of the jaw members 451, which can facilitate the manufacturing process. For example, such an assembly process allows an axel that forms the pin members 473 to be secured (e.g., welded) to the remainder of the actuation link 475 while positioned outside, rather than inside, of the clevis 470 and jaw members 451.

The end effector 430 may be configured as an electrosurgical instrument, such as a vessel sealer. In particular, the electrode 453 may deliver electrosurgical energy to perform operations such as sealing tissue (e.g., a blood vessel) grasped between the jaw members 451 whereupon the cutting element 480 may be driven to translate relative to the still-closed jaw members 451 to cut the now-sealed vessel. The cutting element 480 may travel within a slot running along a length of the jaw member 451. As shown in FIG. 5, the slot may comprise (or be formed from) a slot 487 in the electrode 453, a slot 486 in the insulating layer 454, and a slot 485 in the jaw base 452. This slot may constrain and guide the cutting element 480 as it translates relative to the jaw members 451. In some embodiments, the slot 485 in the jaw base 452 may be among the features at the distal end of the jaw base 452 that can be relatively small and intricate to form in the jaw base 452 when the dimensions thereof are shrunk. In particular, and end of the slot 485 at a distal end thereof may be relatively more difficult to form than more proximal portions. However, in the embodiment of FIGS. 4-8B, the distal end of the jaw base 452 may be omitted, thus allowing for this relatively more difficult portion of the slot 485 of the jaw base 452 to also be omitted. Thus, the jaw base 452 can have its dimensions shrunk without significantly increasing the difficulty in its manufacture.

Turning now to FIG. 9, an embodiment of a computer-assisted instrument control system 1000 for remote control of instruments will be described. FIG. 9 is a schematic block diagram of the computer-assisted instrument control system 1000 for remote control of instruments. The system 1000 comprises a manipulator assembly 1100, a control system 1106, and a user input and feedback system 1104. The system 1000 may also include an auxiliary system 1108. These components of the system 1000 are described in greater detail blow.

The manipulator assembly 1110 comprises one or more manipulators 1114. FIG. 9 illustrates three manipulators 1114, but any number of manipulators 1114 may be included. In the embodiment of FIG. 9, each manipulator 1114 comprises a kinematic structure of two or more links 1115 coupled together by one or more joints 1116. The joints 1116 may impart various degrees of freedom of movement to the manipulator 1114, allowing the manipulator 1114 to be moved around a workspace. For example, some joints 1116 may provide for rotation of links 1115 relative to one another, other joints 1116 may provide for translation of links 1115 relative to one another, and some may provide for both rotation and translation. Some or all of the joints 1116 may be powered joints, meaning a powered drive element may control movement of the joint 1116 through the supply of motive power. Such drive elements may comprise, for example, electric motors, pneumatic or hydraulic actuators, etc. Additional joints 1116 may be unpowered joints. In addition to drive elements that control the joints 1116, the manipulator 1114 may also include drive elements (not illustrated) that drive inputs of the instrument 1102 to control operations of the instrument, such as moving an end-effector of the instrument, opening/closing jaws, driving translating and/or rotating components, etc. In some embodiments, the manipulator assembly can include flux delivery transmission capability as well, such as, for example, to supply electricity, fluid, vacuum pressure, light, electromagnetic radiation, etc. to the end effector. In other embodiments, such flux delivery transmission may be provided to an instrument through another auxiliary system, described further below. FIG. 9 illustrates each manipulator 1114 as having two links 1115 and one joint 1116, but in practice a manipulator may include more links 1115 and more joints 1116, depending on the needs of the system 1000.

Each manipulator 1114 may be configured to support and/or operate one or more instruments 1102. In some examples the instruments 1102 may be fixedly coupled to the manipulator 1114, while in other examples one of the links 1115 may be configured to have one or more separate instruments 1102 removably coupled thereto. The instruments 1102 may include any tool or instrument, including for example industrial instruments and medical instruments (e.g., surgical instruments, imaging instruments, diagnostic instruments, therapeutic instruments, etc.). The instrument 100 described above may be used as any one of the instruments 1102.

The system 1000 can also include a user input and feedback system 1104 operably coupled to the control system 106. The user input and feedback system 1104 comprises one or more input devices to receive input control commands to control operations of the manipulator assembly 1110. Such input devices may include but are not limited to, for example, telepresence input devices, triggers, grip input devices, buttons, switches, pedals, joysticks, trackballs, data gloves, trigger-guns, gaze detection devices, voice recognition devices, body motion or presence sensors, touchscreen technology, or any other type of device for registering user input. In some cases, an input device may be provided with the same degrees of freedom as the associated instrument that they control, and as the input device is actuated, the instrument, through drive inputs from the manipulator assembly, is controlled to follow or mimic the movement of the input device, which may provide the user a sense of directly controlling the instrument. Telepresence input devices may provide the operator with telepresence, meaning the perception that the input devices are integral with the instrument. The user input and feedback system 1104 may also include feedback devices, such as a display device (not shown) to display images (e.g., images of the worksite as captured by one of the instruments 1102), haptic feedback devices, audio feedback devices, other graphical user interface forms of feedback, etc.

The control system 1106 may control operations of the system 1000. In particular, the control system 1106 may send control signals (e.g., electrical signals) to the manipulator assembly 1110 to control movement of the joints 1116 and to control operations of the instruments 1102 (e.g., through drive interfaces at the manipulators 1114). In some embodiments, the control system 1106 may also control some or all operations of the user input and feedback system 1104, the auxiliary system 1108, or other parts of the system 1000. The control system 1106 may include an electronic controller to control and/or assist a user in controlling operations of the manipulator assembly 1110. The electronic controller comprises processing circuitry configured with logic for performing the various operations. The logic of the processing circuitry may comprise dedicated hardware to perform various operations, software (machine readable and/or processor executable instructions) to perform various operations, or any combination thereof. In examples in which the logic comprises software, the processing circuitry may include a processor to execute the software instructions and a memory device that stores the software. The processor may comprise one or more processing devices capable of executing machine readable instructions, such as, for example, a processor, a processor core, a central processing unit (CPU), a controller, a microcontroller, a system-on-chip (SoC), a digital signal processor (DSP), a graphics processing unit (GPU), etc. In examples in which the processing circuitry includes dedicated hardware, in addition to or in lieu of the processor, the dedicated hardware may include any electronic device that is configured to perform specific operations, such as an Application Specific Integrated Circuit (ASIC), a Field Programmable Gate Array (FPGA), a Complex Programmable Logic Device (CPLD), discrete logic circuits, a hardware accelerator, a hardware encoder, etc. The processing circuitry may also include any combination of dedicated hardware and processor plus software.

Differing degrees of user control versus autonomous control may be utilized in the system 1000, and embodiments disclosed herein may encompass fully user-controlled systems, fully autonomously-controlled systems, and systems having any combination of user and autonomous control. For operations that are user-controlled, the control system 1106 generates control signals in response to receiving a corresponding user input command via the user input and feedback system 1104. For operations that are autonomously controlled, the control system 1106 may execute pre-programmed logic (e.g., a software program) and may determine and send control commands based on the programming (e.g., in response to a detected state or stimulus specified in the programming). In some systems, some operations may be user controlled and others autonomously controlled. Moreover, some operations may be partially user controlled and partially autonomously controlled-for example, a user input command may initiate performance of a sequence of events, and then the control system 1106 may perform various operations associated with that sequence without needing further user input.

The auxiliary system 1108 may comprise various auxiliary devices that may be used in operation of the system 1000. For example, the auxiliary system 1108 may include power supply units, auxiliary function units (e.g., functions such as irrigation, evacuation, energy supply, illumination, sensors, imaging, etc.). As one example, in a system 1000 for use in a medical procedure context, the auxiliary system 1108 may comprise a display device for use by medical staff assisting a procedure, while the user operating the input devices may utilize a separate display device that is part of the user input and feedback system 1104. As another example, in a system 1000 for use in a medical context, the auxiliary system 1108 may comprise flux supply units that provide surgical flux (e.g., electrical power) to instruments 1102. An auxiliary system 1108 as used herein may thus encompass a variety of components and does not need to be provided as an integral unit.

As noted above, one or more instruments 1102 can be mounted to the manipulator 1114. In some embodiments, an instrument carriage physically supports the mounted instrument 1102 and has one or more actuators (not illustrated) to provide driving forces to the instrument 1102 to control operations of the instrument 1102. The actuators may provide the driving forces by actuating drive outputs (not illustrated), such as rotary disc outputs, joggle outputs, linear motion outputs, etc. The drive outputs may interface with and mechanically transfer driving forces to corresponding drive inputs of the instrument 1102 (directly, or via intermediate drive outputs, which may be part of a sterile instrument adaptor (ISA) (not illustrated)). The ISA may be placed between the instrument 1102 and the instrument carriage to maintain sterile separation between the instrument 1102 and the manipulator 114. The instrument carriage may also comprise other interfaces (not illustrated), such as electrical interfaces to provide and/or receive electrical signals to/from the instrument 1102.

The embodiments described herein may be well suited for use in medical applications. In particular, some embodiments are suitable for use in, for example, surgical, teleoperated surgical, diagnostic, therapeutic, and/or biopsy procedures. Such procedures could be performed, for example, on human patients, animal patients, human cadavers, animal cadavers, and portions or human or animal anatomy. Some embodiments may also be suitable for use in, for example, for non-surgical diagnosis, cosmetic procedures, imaging of human or animal anatomy, gathering data from human or animal anatomy, training medical or non-medical personnel, and procedures on tissue removed from human or animal anatomies (without return to the human or animal anatomy). Even if suitable for use in such medical procedures, the embodiments may also be used for benchtop procedures on non-living material and forms that are not part of a human or animal anatomy. Moreover, some embodiments are also suitable for use in non-medical applications, such as industrial robotic uses, including, but not limited to, sensing, inspecting, and/or manipulating non-tissue work pieces. In non-limiting embodiments, the techniques, methods, and devices described herein may be used in, or may be part of, a computer-assisted surgical system employing robotic technology such as the da Vinci® Surgical Systems commercialized by Intuitive Surgical, Inc., of Sunnyvale, California. Those skilled in the art will understand, however, that aspects disclosed herein may be embodied and implemented in various ways and systems, including manually operated instruments and computer-assisted, teleoperated systems, in both medical and non-medical applications. Reference to the daVinci® Surgical Systems are illustrative and not to be considered as limiting the scope of the disclosure herein.

It is to be understood that both the general description and the detailed description provide example embodiments that are explanatory in nature and are intended to provide an understanding of the present disclosure without limiting the scope of the present disclosure. Various mechanical, compositional, structural, electrical, and operational changes may be made without departing from the spirit and scope of this description and the claims. Further, the terminology used herein to describe aspects of the invention, such as spatial and relational terms, is chosen to aid the reader in understanding example embodiments of the invention but is not intended to limit the invention. For example, spatially terms—such as “beneath”, “below”, “lower”, “above”, “upper”, “proximal”, “distal”, “up”, “down”, and the like—may be used herein to describe directions or one element's or feature's spatial relationship to another element or feature as illustrated in the figures. These spatial terms are used relative to the figures and are not limited to a particular reference frame in the real world. Thus, for example, the direction “up” in the figures does not necessarily have to correspond to an “up” in a world reference frame (e.g., away from the Earth's surface). Furthermore, if a different reference frame is considered than the one illustrated in the figures, then the spatial terms used herein may need to be interpreted differently in that different reference frame. For example, the direction referred to as “up” in relation to one of the figures may correspond to a direction that is called “down” in relation to a different reference frame that is rotated 180 degrees from the figure's reference frame. As another example, if a device is turned over 180 degrees in a world reference frame as compared to how it was illustrated in the figures, then an item described herein as being “above” or “over” a second item in relation to the Figures would be “below” or “beneath” the second item in relation to the world reference frame. Thus, the same spatial relationship or direction can be described using different spatial terms depending on which reference frame is being considered. Moreover, the poses of items illustrated in the figure are chosen for convenience of illustration and description, but in an implementation in practice the items may be posed differently.

As used herein, “proximal” and “distal” are spatial/directional terms that describe locations or directions based on their relative location in a kinematic chain. In the context of the present disclosure, the directions proximal and distal are labeled relative to the instrument in various figures, with proximal describing the direction along the instrument toward the force transmission system and distal describing the direction along the instrument toward the end effector. As such, the proximal and distal directions are not fixed in space, but rather are used herein to describe different end portions of the instrument itself regardless of its specific orientation in space.

In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components, unless specifically noted otherwise. Mathematical and geometric terms are not necessarily intended to be used in accordance with their strict definitions unless the context of the description indicates otherwise, because a person having ordinary skill in the art would understand that, for example, a substantially similar element that functions in a substantially similar way could easily fall within the scope of a descriptive term even though the term also has a strict definition.

Elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment.

Further modifications and alternative embodiments will be apparent to those of ordinary skill in the art in view of the disclosure herein. For example, the devices and methods may include additional components or steps that were omitted from the diagrams and description for clarity of operation. Accordingly, this description is to be construed as illustrative only and is for the purpose of teaching those skilled in the art the general manner of carrying out the present disclosure. It is to be understood that the various embodiments shown and described herein are to be taken as exemplary. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. In some instances, well-known structures, systems, and techniques have not been shown or described in detail in order not to obscure the embodiments. Changes may be made in the elements described herein without departing from the scope of the present teachings and following claims.

Other embodiments in accordance with the present disclosure will be apparent to those skilled in the art from consideration of the specification and practice of the invention disclosed herein. It is intended that the specification and examples be considered as exemplary only, with the following claims being entitled to their fullest breadth, including equivalents, under the applicable law.

1. An instrument comprising:
a shaft comprising a proximal end portion and a distal end portion; and
an end effector coupled to the distal end portion of the shaft, the end effector comprising a first jaw member and a second jaw member opposing each other, each of the first and second jaw members extending distally relative to the shaft and movable relative to one another between an open configuration and a closed configuration,
wherein each of the first and second jaw members comprises:
a proximal connection portion coupled to the distal end portion of the shaft,
a jaw base extending distally from the proximal connection portion, the jaw base terminating at a distal end,
an electrode coupled to the jaw base and comprising an exposed contact surface configured to contact an object between the first and second jaw members in a closed configuration of the first and second jaw members, the electrode having a distal portion extending distally beyond the distal end of the jaw base and terminating in a distal end, and
a support feature configured to support the distal portion of the electrode, the support feature located distal to the distal end of the jaw base and on a side of the electrode opposite the exposed contract surface, and
wherein a thickness of each of the first and second jaw members, measured along a height dimension perpendicular to the contact surface, is less in a region distal to the distal end of the jaw base than in a region including the jaw base.

2. The instrument of claim 1,
wherein the support feature comprises a portion of the electrode extending perpendicularly from the contact surface in a direction away from the opposing jaw member.

3. The instrument of claim 2,
wherein the support feature comprises an apex located proximate the distal end of the jaw base and an angled edge extending distally from the apex toward the contact surface.

4. The instrument of claim 2,
wherein the electrode comprises a side flange that extends from the contact surface in a direction away from the opposing jaw member, and
wherein the support feature is part of the side flange and protrudes further in the direction away from the opposing jaw member than a remainder of the side flange.

5. The instrument of claim 2,
wherein each of the first and second jaw members further comprises a second support feature and an insulation layer disposed between the electrode and the jaw base; and
wherein the second support feature comprises a distal portion of the insulation layer having a thickness, measured along the height dimension of the jaw member, larger than a thickness of a remainder of the insulation layer, the distal portion of the insulation layer extending distally beyond the distal end of the jaw base.

6. The instrument of claim 1,
wherein each of the first and second jaw members further comprises an insulation layer disposed between the electrode and the jaw base; and
wherein the support feature comprises a distal portion of the insulation layer having a thickness, measured along the height dimension of the jaw member, larger than a thickness of a remainder of the insulation layer, the distal portion of the insulation layer extending distally beyond the distal end of the jaw base.

7. An instrument comprising:
a shaft comprising a proximal end portion and a distal end portion; and
an end effector coupled to the distal end portion of the shaft, the end effector comprising:
a first jaw member and a second jaw member opposing and pivotably coupled to each other by pivot pin members; and
an actuation link engaged with the first and second jaw members to drive the first and second jaw members to pivotably move between open and closed states;
wherein each of the first and second jaw members comprises a long tang and a short tang, the long tang being longer than the short tang,
wherein each of the long and short tangs comprises a pivot aperture engaged with one of the pivot pin members, and
wherein the long tang comprises a ramped slot engaged with the actuation link.

8. The instrument of claim 7,
wherein the short tang of each of the first and second jaw members lacks a ramped slot.

9. The instrument of claim 7,
wherein the short tang of each of the first and second jaw members terminates at a position distal of the ramped slot of the long tang of each of the first and second jaw members.

10. The instrument of claim 7,
wherein each of the first and second jaw members comprises a grasping portion coupled to and extending distally from the long and short tangs, the grasping portion configured to grasp an object positioned between the first and second jaw members in the closed state of the first and second jaw members.

11. The instrument of claim 10,
wherein the grasping portion of each of the first and second jaw members further comprises an electrode.

12. The instrument of claim 11,
wherein the grasping portion of each of the first and second jaw members further comprises a jaw base and a support feature,
wherein the electrode is coupled to the jaw base and has a distal portion extending distally beyond the jaw base, and
wherein the support feature is configured to support the distal portion of the electrode.

13. The instrument of claim 12,
wherein the electrode comprises a contact surface configured to contact material grasped between the first and second jaw members, and
wherein the support feature comprises a portion of the electrode extending from the contact surface in a direction away from the opposing jaw member.

14. The instrument of claim 13,
wherein each of the first and second jaw members further comprises a second support feature and an insulation layer disposed between the electrode and the jaw base; and
wherein the second support feature comprises a distal portion of the insulation layer having a thickness larger than a thickness of a remainder of the insulation layer along a height dimension of the jaw member, the distal portion of the insulation layer extending distally beyond a distal end of the jaw base.

15. The instrument of claim 12,
wherein each of the first and second jaw members further comprises an insulation layer disposed between the electrode and the jaw base; and
wherein the support feature comprises a distal portion of the insulation layer having a thickness larger than a thickness of a remainder of the insulation layer along a height dimension of the jaw member, the distal portion of the insulation layer extending distally beyond a distal end of the jaw base.

16. The instrument of claim 7,
wherein the respective long and short tangs of the first and second jaw members are interleaved.

17. The instrument of claim 7, further comprising:
a clevis coupling the first and second jaw members to the shaft,
wherein the pivot pin members engage with apertures of the clevis to pivotably couple the first and second jaw members to the clevis,
wherein the actuation link is engaged with guide slots in the clevis configured to constrain motion of the actuation link to translation relative to the clevis, and
wherein the actuation link is configured to translate along the guide slots and cause pivoting of the first and second jaw members via interaction between the actuation link and the ramped slots.

19. canceled

20. A method of assembling a jaw mechanism of an instrument end effector, comprising:
providing a first jaw member comprising two first pivot apertures and a first ramped slot;
providing a second jaw member comprising two second pivot apertures and a second ramped slot;
coupling an actuation link to an actuation element, the actuation link comprising two
engaging the pin members of the actuation links with the first and second ramped slots by moving the first and second jaw members laterally towards one another;
pivoting the first and second jaw members relative to one another while the pin members of the actuation link are engaged with the first and second ramped slots so as to bring the respective first and second pivot apertures of the first and second jaw members into alignment with one another; and
inserting pivot pin members through the aligned first and second pivot apertures of the first and second jaw members.

21. The method of claim 20,
wherein each of the first and second jaw members comprises a long tang and a short tang, the long and short tangs each comprising one of the first and second pivot apertures;
wherein the method further comprises, prior to moving the first and second jaw members laterally towards one another, angling the first and second jaw members relative to one another such that the short tangs of each of the first and second jaw members do not interfere with one another as the first and second jaw members are moved laterally towards one another.

22. The method of claim 20, further comprising engaging the pin members of the actuation link with first and second guide slots in a clevis such that motion of the actuation link is constrained to translation relative to the clevis.
